(19) United States
(12) Patent Application Publication
(10) Pub. No.: US 2011/0307010 A1
(43) Pub. Date: Dec. 15, 2011

(54) INTERSPINOUS DEVICE AND METHOD OF IMPLANTING

(75) Inventor: Ben Pradhan, La Canada Flintridge, CA (US)

(73) Assignee: OSPREY BIOMEDICAL CORP., Manhattan Beach, CA (US)

(21) Appl. No.: 12/868,288

(22) Filed: Aug. 25, 2010

Related U.S. Application Data
(60) Provisional application No. 61/305,897, filed on Feb. 18, 2010.

(51) Int. Cl. A61B 17/70

(52) U.S. Cl. 606/249

(57) ABSTRACT

In an interspinous device made of an allograft, the device is machined to define a substantially rectangular structure with flat major surfaces and rounded upper and lower notches for engaging the spinous processes of adjacent vertebrae and provide consistent structures that abut multiple surfaces of the spine.

### FIELD OF THE INVENTION

The invention relates to medical implants. In particular it relates to interspinous devices for treating spinal stenosis.

### BACKGROUND OF THE INVENTION

Interspinous devices are structural members for treating a condition known as spinal stenosis, which is a narrowing of the spinal canal (a bony tunnel that surrounds the spinal cord) and resulting in lower back pain or leg pain.

This is best appreciated by considering FIG. 1, which shows a side view of part of a human spine 100 with its vertebral bones (vertebrae) 102 and disks 104. The posterior or back side of the vertebral bones 102 includes a posteriorly extending bone structure known as the spinous process 106. This is, seen from the top in FIG. 2, which shows a vertebral bone (vertebra) 200 with its spinous process 202.

The interspinous devices are designed to open up the foramen (also referred to as the foraminal canal), where the nerve endings pass outward from the central spinal region at each level of the spine between the vertebrae. The spinal region includes the spinal canal 204 surrounding the spinal cord (not shown).

FIG. 3 is a section through a portion of a human spine 300 along the mid-sagittal plane, showing the vertebral body (with its vertebrae 300 and disks 302) on the right and the spinous process 304 on the left. In this case the interspinous process space 306 has collapsed. The spinous processes 304 of adjacent vertebrae therefore come together to create a pinching effect on the interspinous process space 306, thereby reducing the space for nerve roots exiting from the spinal region 308 through the foramen. It may also cause a partial collapse or narrowing of the spinal canal as indicated by the narrowing 308, creating a pressure on the spinal cord.

It will therefore be appreciated that backward bending of the spine exacerbates the problem by further reducing the space available for nerve roots exiting the foramen, as shown in FIG. 3, typically causing the patient increased back pain.

The interspinous devices therefore act as a spacer to provide the nerve roots with more space to exit the foraminal openings. One prior art device, discussed in US patent application publication number 2010/0076492 to Warner makes use of a cylindrical spacer that is laterally inserted by making a lateral incision in the spinous process ligament. The spacer is deployed by adequately distracting the collapsed interspinous process space by inserting a dilator. In the Warner device a rotation cannula is place over the dilator and acts as a housing for receiving the spacer and allowing it to be rotated from a plane parallel to the spinous processes to a plane perpendicular to the spinous processes, whereafter the dilator and rotation cannula are removed.

The problem with spacers such as the Warner spacer discussed above is that it engages only with the upper and lower spinous processes and therefore provides few points of contact and a reduced ability for fusion with the bone.

### SUMMARY OF THE INVENTION

According to the invention there is provided an interspinous device or spacer, comprising a body defining an anterior surface and a planar posterior surface, an upper surface defining a first interspinous process-engaging notch, and a lower surface defining a second interspinous process-engaging notch.

The anterior surface is preferably also planar.

The first and second interspinous process-engaging notches may comprise concave notches. The first interspinous process-engaging notch may have a radius that is greater than that of the second interspinous process-engaging notch.

The body may define a left surface and a right surface, and both the left and right surfaces may define a notch. Both of the notches defined by the left and right surfaces may comprise a U-shaped notch with parallel side walls and a substantially flat connecting base for engaging a holder during insertion of the interspinous device.

The spacer body may include two or more holes extending through the body from the posterior surface to the anterior surface.

All of the surfaces of the body are preferably machined from natural bone, e.g., allograft.

Further, according to the invention, there is provided an interspinous device or spacer, comprising a body defining an anterior surface and a posterior surface, an upper surface defining a first interspinous process-engaging notch, and a lower surface defining a second interspinous process-engaging notch, the notches being concave in shape.

The first interspinous process-engaging notch preferably has a larger radius than the second interspinous process-engaging notch. The posterior and anterior surfaces are preferably planar.

The spacer body may include two or more holes extending through the body from the posterior surface to the anterior surface.

Still further, according to the invention, there is provided a method of treating a collapsed interspinous process space between adjacent vertebrae of a patient, comprising opening the collapsed space, and inserting an interspinous spacer between interspinous processes of adjacent vertebra, the spacer comprising a substantially rectangular plate-like structure with posterior and anterior major surfaces and upper and lower spinous process-engaging surfaces with concave notches for engaging the spinous processes. The opening of the collapsed space may comprise at least one of positioning the patient to distract the vertebrae from one another, and using distracting tools. The method may further comprise securing a plate across the spacer to prevent it from being pushed out. The vertebrae may include lamina regions and articular processes, and the step of inserting the spacer may include bringing the anterior major surface of the spacer into abutment with at least one of the lamina regions of the vertebrae, and the articular processes of the vertebrae.

### BRIEF DESCRIPTION OF THE DRAWINGS

FIG. 1 shows a side view of part of a typical human spine;

FIG. 2 shows a top view of a human vertebra;

FIG. 3 shows a section through part of a human spine along the mid-sagittal plane;

FIG. 4 shows a three dimensional view of one embodiment of an interspinous device or spacer of the invention;

FIG. 5 shows a side view of the spacer of FIG. 4;

FIG. 6 shows a bottom view of the spacer of FIG. 5;

FIG. 7 shows a side view of part of a typical human spine with one embodiment of a spacer of the invention inserted between two adjacent vertebrae;

FIG. 8 shows a top view of a human vertebra with one embodiment of a spacer of the invention place on top of the spinous process of the vertebra;

FIG. 9 shows a three dimensional view of another embodiment of an interspinous device or spacer of the invention;

FIG. 10 shows a side view of the spacer of FIG. 9;

FIG. 11 shows a three dimensional view of yet another embodiment of an interspinous device or spacer of the invention; and

FIG. 12 is a side view of a portion of a spine showing a spacer of the invention in position relative to a pair of vertebrae and with a securing plate securing the spacer to the vertebrae.

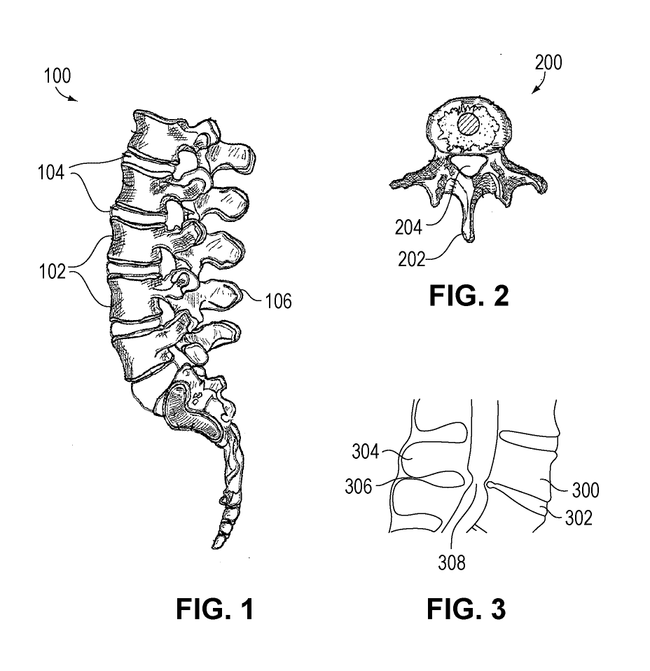

### DETAILED DESCRIPTION OF THE INVENTION

For purposes of this application the following terms will have the meanings as described below:

The term “allograft”, refers to a graft taken from a different individual of the same species.

The term “sagittal plane” refers to a plane that splits the body into left and right segments. The mid-sagittal or median plane splits the body into equal left and right halves.

The term “coronal plane” refers to a plane that splits the body into anterior and posterior segments.

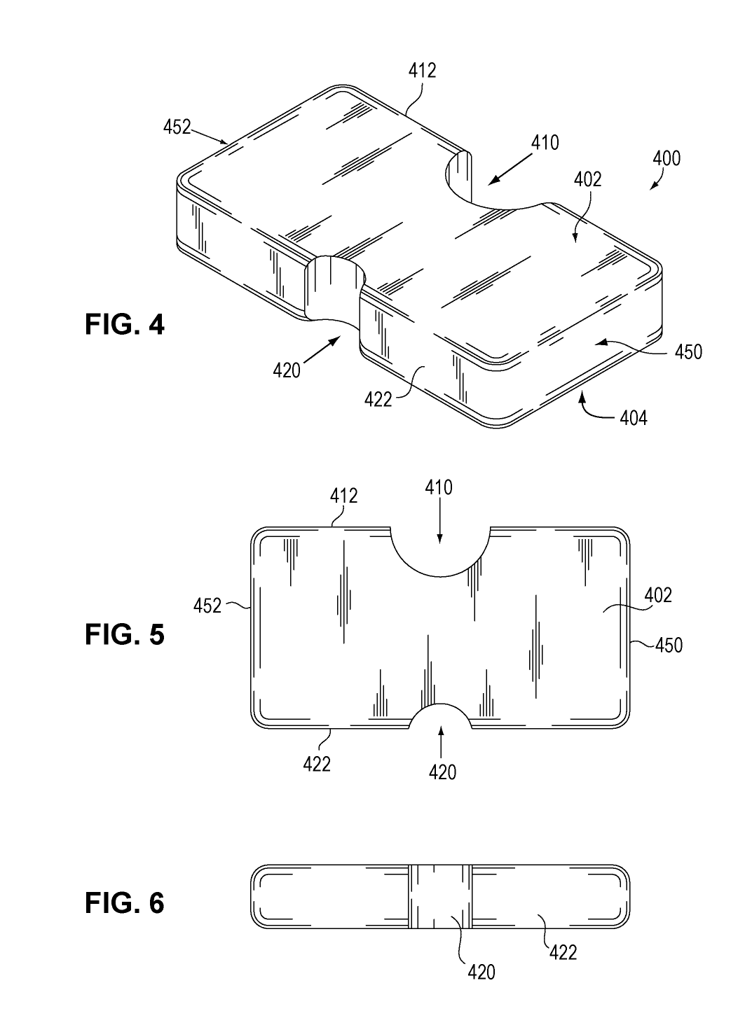

One embodiment of an interspinous device or spacer of the invention is shown in FIGS. 4 to 6. The spacer 400 includes a body made from a biological material such as an allograft. The spacer 400, in practice is inserted between two adjacent spinous processes in which the interspinous process space has collapsed. The spacer 400 will therefore be located on the posterior side of the vertebral body.

The spacer 400 defines a posterior surface 402, which in its operative orientation, faces away from the vertebral body and has a substantially flat or planar surface for retaining the spacer using a plate, as is discussed in greater detail below. The spacer also defines an anterior surface 404 that faces the vertebral body once the spacer has been inserted.

In order to correctly position the spacer 400 between the spinous processes and to help secure the spacer, a concave notch 410 is formed into an operatively upper surface 412, and a concave notch 420 is formed into an operatively lower surface. As is shown in FIGS. 4 and 5, the upper notch 410 has a larger radius than the lower notch 420 in this embodiment, in order to better accommodate the natural anatomy of the spinous processes that are received in the upper and lower notches 410, 420.

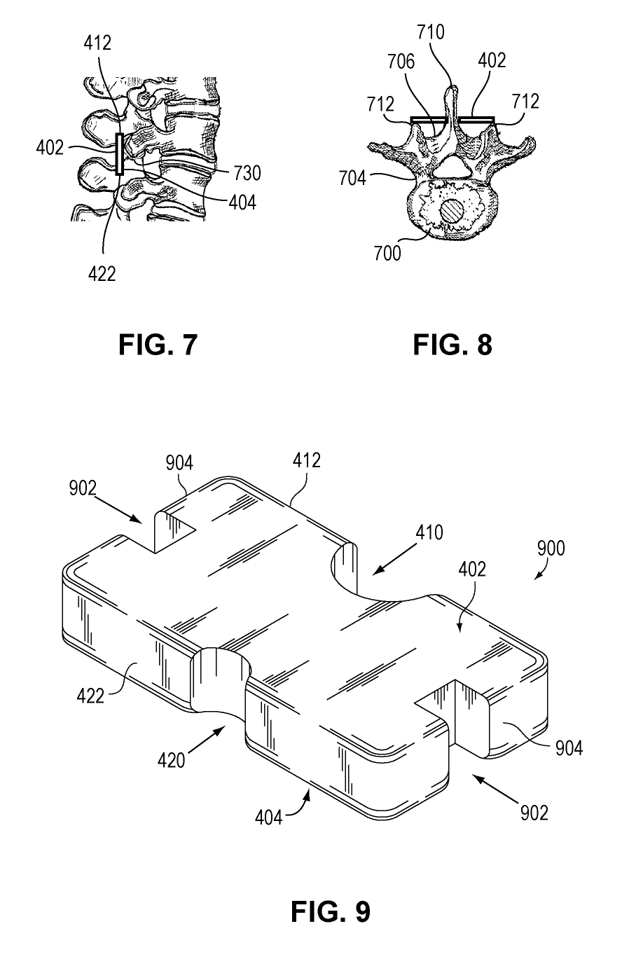

In practice the spacer 400 is positioned between two spinous processes by sliding the upper notch 410 under and up the lower surface of the top spinous process as shown in FIGS. 7 and 8.

Each vertebra includes a body 700 defining the anterior portion of the spinal canal 702. The spinal canal 702 is bounded on its sides by cylindrical bony projections 704, known as pedicles, and by the lamina 706, a bony connector between the spinous process 710 and the articular process 712.

The articular facets comprise pairs of upper and lower joint sections known as the superior and inferior articular facets indicated in FIG. 7 by the joint 730.

The body of the spacer extends laterally to abut not only the lower surface 720 of the top spinous process, and the upper surface 722 of the bottom spinous process (with its notches 410, 412), but to also abut at least the lamina 706 or the articular facets 712, thereby providing a larger number of points of contact and more locations for fusion with the bone.

All of the surfaces of the spacer (posterior surface 402 and anterior surfaces 404, upper surface 412 and lower surface 422 with their notches 410, 420, and the side surfaces 430, 432 are machined to achieve consistency between spacers. This contrasts with many prior art spacers that make use of the natural anatomy of bone shafts which often are irregular/uneven, thus resulting in inconsistent shapes or size, which results in less surface contact to with the spinous processes, lamina and facets since the irregular shape is likely to wobble and potentially fail to fuse. As well, some of the prior art have a few slots cut into the bone, but again are inconsistent and require much surgeon manipulation to make the spacer fit against the spinous process. The surgeon using such prior art spacers is thus faced with a different looking spacer each time he/she opens a package.

One feature of the invention is the provision of flat, machined anterior and posterior surface, rather than concave or convex surfaces as defined by the natural contours of the bone, which is commonly found in prior art spacers that make use of natural bone material for their spacers.

By machining the allograft material to define a rectangular structure with all of its surfaces machined, consistent spacers can be provided, in accordance with one aspect of the present invention. The flat anterior major surface 404 ensures that the surface abuts the lamina and articular facets, thereby promoting a solid fusion. In contrast, a concave or convex major anterior surface would provide less surface to surface contact and therefore less fusion area.

The flat posterior major surface 402 provides a flat abutment surface for a plate insofar as a fixation plate is secured across the spacer. The flat posterior surface avoids the plate from rocking and provides a solid fixation for fusion.

In practice, the surgeon typically slides the upper surface 412 of the spacer under the upper spinous process 750 and then pivots the lower surface of the spacer into place over the lower spinous process 752 to seat the upper notch 410 (FIG. 4) into the lower surface of the upper spinous process, and the lower notch 420 in the upper surface of the lower spinous process, as shown in FIG. 7. Alternatively, the surgeon first inserts the lower surface of the spacer over the lower spinous process 752 and then pivots the upper surface of the spacer into place under the upper spinous process.

The upper and lower notches 410, 420 are rounded, and in this embodiment form semi-circles. The radius of the upper notch is chosen to be larger than the lower notch and both are dimensioned to allow the spacer to be guided between the natural contours of the spinous processes more easily.

As shown in FIGS. 7 and 8, the spacer defines an anterior major surface 404 that is flat to allow it to lie snugly against the lamina regions 706 and the articular processes 712 of the vertebrae. The posterior major surface 402 of the spacer is preferably also planar to allow a securing plate (not shown) to be secured across the spacer and avoid the spacer wobbling relative to the plate. The use of a securing plate is discussed in greater detail below.

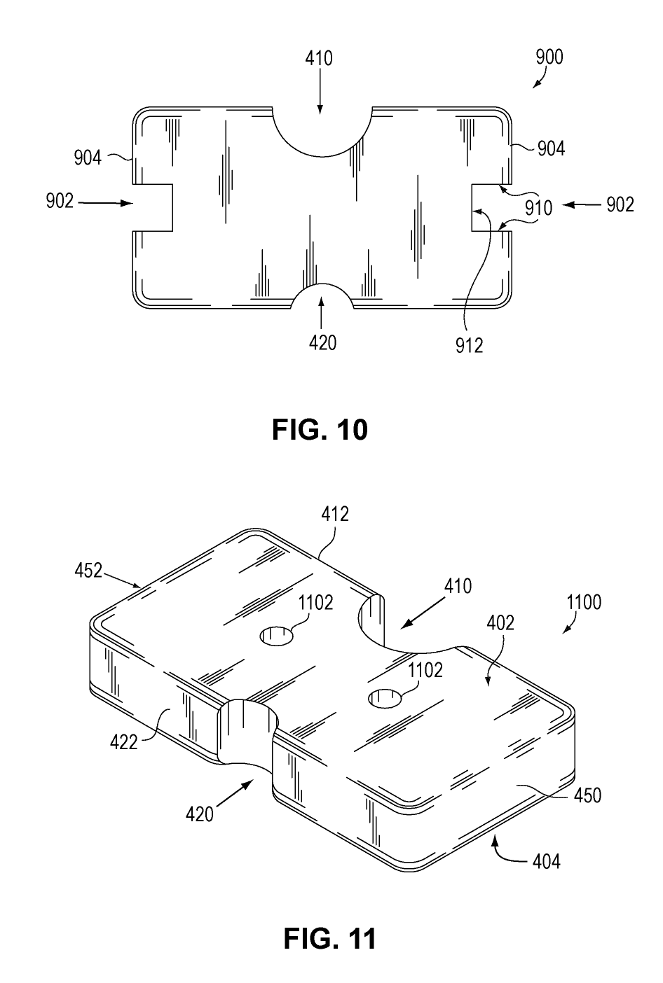

Another embodiment of a spacer of the invention is shown in FIGS. 9 and 10, which show a three dimensional view and front view, respectively. The spacer 900 is similar to the spacer 400. The same reference numerals will therefore be used to depict similar regions of the spacer. The spacer 900 however also includes U-shaped notches 902 in the side surfaces 904.

The U-shaped notches 902 in this embodiment have parallel side-walls 910 and a flat connecting base 912, and provide gripping surfaces for a tool to grip the spacer during insertion between the spinous processes.

Yet another embodiment of a spacer of the invention is shown in FIG. 11. This embodiment is again similar to the embodiment of FIG. 4 and similar regions have been depicted using the same reference numerals. However, in the spacer 1100 two holes 1102 extend through the spacer from the posterior surface 402 to the anterior surface 404. The holes provide an alternative method of grasping the spacer for purposes of insertion.

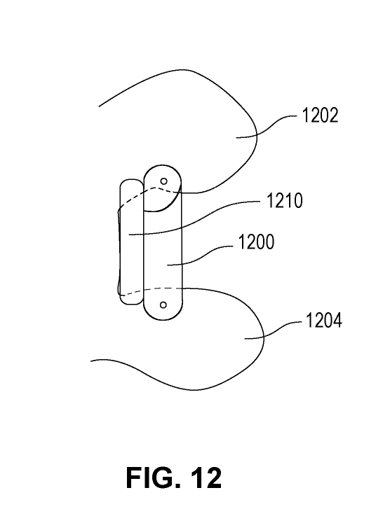

In all of the embodiments of the invention, a securing plate 1200 is preferable screwed into the upper and lower spinous processes 1202, 1204 across the spacer 1210 as shown in FIG. 12. This avoids the spacer from being pushed out when pressure is exerted by the spinous processes and the lamina and articular processes. It will be appreciated that any suitable plate or clamp currently used in the industry could be used to hold the spacer in place.

One of the advantages of the spacers of the present invention is that they are highly machined unlike prior art devices. This ensures a flat surface for abutting the plate that holds the spacer in place, which ensures that there is no “rocking” or wobbling” of the plate relative to the spacer.

While the spacer was described with respect to a few specific embodiments, it will be appreciated that the invention is not so limited, but includes other embodiments within the scope of the claims.

What is claimed is:

1. An interspinous spacer, comprising:
a body defining an anterior surface and a planar posterior surface;
an upper surface defining a first interspinous process-engaging notch; and
a lower surface defining a second interspinous process-engaging notch.

2. An interspinous spacer of claim 1, wherein the anterior surface has a planar configuration.

3. An interspinous spacer of claim 1, wherein the first and second interspinous process-engaging notches comprise concave notches.

4. An interspinous spacer of claim 3, wherein the first interspinous process-engaging notch has a radius that is greater than that of the second interspinous process-engaging notch.

5. An interspinous spacer of claim 4, wherein the body defines a left surface and a right surface, and both the left and right surfaces define a notch.

6. An interspinous spacer 4, wherein the spacer body includes two or more holes extending through the body from the posterior surface to the anterior surface.

7. An interspinous spacer of claim 1, wherein the spacer is machined from natural bone.

8. An interspinous spacer, comprising:
an anterior surface;
a posterior surface;
an upper surface defining a first interspinous process-engaging notch; and
a lower surface defining a second interspinous process-engaging notch, the notches being concave in shape.

9. An interspinous spacer of claim 8, wherein the first and second interspinous process-engaging notches each have a semicircular portion, the semicircular portion of the first interspinous process-engaging notch having a larger radius than the semicircular portion of the second interspinous process-engaging notch.

10. An interspinous spacer of claim 9, wherein the posterior and anterior surfaces are planar.

11. An interspinous spacer of claim 10, further comprising two or more holes extending from the posterior surface to the anterior surface.

12. A method of treating a collapsed interspinous process space between adjacent vertebrae of a patient, comprising:
opening the collapsed space; and
inserting an interspinous spacer between interspinous processes of adjacent vertebra, the spacer comprising a substantially rectangular plate-like structure with posterior and anterior major surfaces and upper and lower spinous process-engaging surfaces with concave notches for engaging the spinous processes.

13. A method of claim 12, wherein the step of opening of the collapsed space comprises at least one of, positioning the patient to distract the vertebrae from one another, and using distracting tools to distract the vertebrae from one another.

14. A method of claim 12, further comprising securing a plate across the spacer to prevent the spacer from being pushed out.

15. A method of claim 14, wherein the vertebrae may include lamina regions and articular processes, and the step of inserting the spacer includes bringing the anterior major surface of the spacer into abutment with at least one of, the lamina regions of the vertebrae, and the articular processes of the vertebrae.
